(19) United States
(12) Patent Application Publication
(10) Pub. No.: US 2012/0053385 A1
(43) Pub. Date: Mar. 1, 2012

(54) METHOD AND DEVICE FOR REDUCING OLEFIN LOSSES DURING THE REMOVAL OF CARBON DIOXIDE FROM AN OLEFIN FLOW FROM DEHYDROGENATION REACTIONS

(75) Inventor: Johannes Menzel, Waltrop (DE)

(73) Assignee: UHDE GMBH, Dortmund (DE)

(21) Appl. No.: 13/256,063

(22) PCT Filed: Feb. 26, 2010
(86) PCT No.: PCT/EP10/01196

§ 371 Date: Sep. 12, 2011

(30) Foreign Application Priority Data
Mar. 12, 2009 (DE) 10 2009 012 452.7

(51) Int. Cl. C07C 5/333; B01D 11/04

(52) U.S. Cl. 585/654; 422/256

(57) ABSTRACT

The product gas from a dehydrogenation reaction is treated by a downstream gas scrub followed by depressurization in a high-pressure flash vessel equipped with mass transfer elements, wherein a fuel gas flows upwardly through the mass transfer elements in the high-pressure flash vessel countercurrent to depressurized solvent so that absorbed hydrocarbons are taken up by the fuel gas. The fuel gas is the heating gas for heating the dehydrogenation reactor and is, for example, natural gas. To increase efficiency, the hydrocarbon stream which has been separated from the acidic gas can be recirculated to the process gas path upstream of the gas scrub.

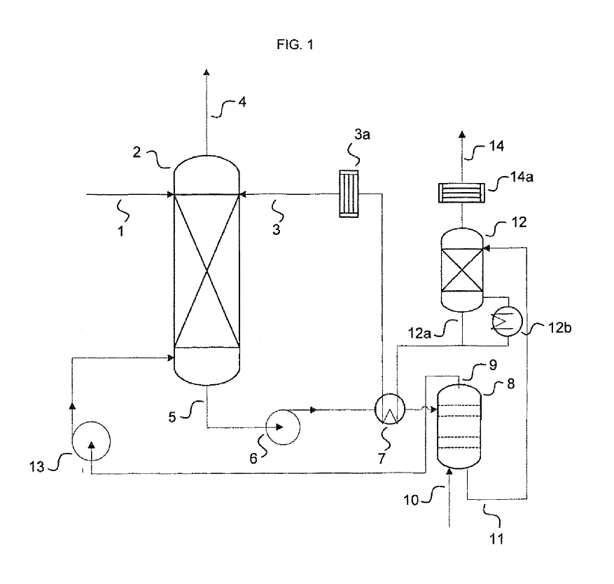

The invention is directed to a process for reducing alkene losses in the dehydrogenation of alkanes to alkenes, in which carbon dioxide is obtained as by-product in the dehydrogenation and the carbon dioxide formed is removed by means of an absorption process from the crude gas formed by the dehydrogenation reaction.

The preparation of olefins and in particular the preparation of gaseous, lower olefins is carried out in industry by dehydrogenation of lower alkanes. In the preparation of olefins, the alkane is mixed with an inert gas, typically with steam, and passed through a dehydrogenation reactor. This is equipped with a catalyst which catalyzes the dehydrogenation of alkanes. The reaction forms the desired alkene which after the reaction is usually present in the product gas in a proportion of a few percent by volume. By-products are formed in the reaction, so that the product gas contains even smaller proportions of carbon dioxide and of carbon dioxide in addition to the desired olefin, the reaction product hydrogen and the inert gas added beforehand.

A typical process for the dehydrogenation of propane to propylene is described in WO 2006050957 A1. Here, a first gas mixture which contains propane, hydrogen and steam as inert gas is fed into at least one catalyst bed having conventional dehydrogenation conditions. Since dehydrogenation reactions are frequently combined with a selective hydrogen combustion by means of which the hydrogen formed is burnt to remove it from the equilibrium, a further propane- and oxygen-containing gas mixture in which the propane content predominates over the oxygen content is introduced into the same reaction apparatus where it reacts with the first gas mixture to form propylene, water vapor and hydrogen. Typical temperatures for carrying out the reaction are from 540° C. to 820° C. at pressures of from 40 to 50 bar.

WO 2006/094938 A2 describes a process for preparing propene from propane, in which a propane-containing feed gas stream is provided and the propane-containing feed gas stream, optionally steam and optionally an oxygen-containing gas stream are fed into a dehydrogenation zone and propane is subjected to dehydrogenation to propene, wherein a product gas stream containing propane, propene, methane, ethane, ethene, nitrogen, carbon monoxide, carbon dioxide, optionally hydrogen and optionally oxygen is obtained and the product gas stream is cooled and optionally compressed and steam is separated off by condensing and a product gas stream depleted in steam is obtained and uncondensable or low-boiling gas constituents are separated off by contacting of the product gas stream with an inert absorption medium and subsequent desorption of the gases dissolved in the inert absorption medium and a C₃-hydrocarbon stream and an offgas stream containing propane, propene, methane, ethane, ethene, nitrogen, carbon monoxide, carbon dioxide, optionally hydrogen and optionally oxygen are obtained by working up the C₃-hydrocarbon stream further. The desorption is carried out by means of a “stripping gas stream” which contains, for example, nitrogen, air, steam or propane/propene mixtures. The use of inexpensive fuel gas, which is typically natural gas, as “stripping gas” is not described.

The inert gas is preferably steam. However, it is also possible to use, for example, carbon dioxide or hydrogen or any further gas which is suitable as inert diluent gas for carrying out the reaction. After the preparative dehydrogenation process, the product olefin obtained is cooled and dried. The drying process can be of any type. The drying process is preferably a condensation process. The hydrogen formed in the dehydrogenation has to be separated off from the desired end product, namely the alkenes, in further process steps. The separation is preferably carried out at relatively low temperatures and elevated pressures.

After removal of the hydrogen, the crude gas is compressed. To keep the loss of alkenes which leave the downstream separation apparatus at the top together with the hydrogen which has been separated off as low as possible, a “cold box” by means of which the hydro-carbons present in the hydrogen, in particular the alkenes, are mostly condensed out is provided. The process gas here experiences a drop in temperature to below −100° C. To prevent carbon dioxide from desubliming in the process gas path at these temperatures, which would lead to blockage of the cold box, it is necessary to remove carbon dioxide from the crude gas to such an extent that formation of solid carbon dioxide cannot occur at any place. Likewise, it is necessary to remove the residual content of sulfur compounds still present, which get into the process with the feed gas, from the crude gas.

For this purpose, the cooled and dried gas is fed to an apparatus which is suitable for removing acidic gas components. This is preferably a column which allows contacting of an acidic gas-absorbing solvent with the gas to be purified under super-atmospheric pressure. The enriched solvent leaving the gas scrubbing column at the bottom is depressurized in a “high-pressure flash vessel” to recover hydrocarbons which have also been absorbed. Most of the acidic gas is carbon dioxide which is partly present in the starting gas and is partly formed from the hydrocarbons in the reaction with the oxygen present in the starting gas.

The solvent which has been partially depressurized in the high-pressure flash vessel is fed to a further separation column in which the acidic gas present in the solvent is separated off from the solvent. This further separation column is usually referred to as solvent regeneration column. The solvent is optionally purified by means of filtration devices and recirculated by means of a circulation pump to the top of the acidic gas absorption column.

The hydrocarbon stream leaving the acidic gas absorption column, which consists of light, gaseous olefins and hydrogen, is fractionated further by means of low-temperature distillation units and freezing-out apparatuses. Examples of low-temperature distillation apparatuses are C₃ splitters and demethanizers. Examples of freezing-out apparatuses are “cold boxes”.

Processes which make it possible to separate off residual carbon dioxide from the high-pressure flash vessel are known. These are, for example, processes according to the prior art which carry out a gas scrub for the hydrocarbon stream. However, these are complicated and costly since they ultimately represent an additional gas scrub. The depressurization carried out in the high-pressure flash vessel is for this reason carried out so that the major part of the absorbed carbon dioxide remains in the solvent.

Since complete separation of the hydrocarbons from the solvent is not possible in the depressurization in the high-pressure flash vessel without the major part of the carbon dioxide also being liberated there, a certain proportion of coabsorbed hydrocarbons gets into the regeneration column in which the acidic gases are virtually completely separated off from the solvent. The hydrocarbons which are also still present in the solvent leave the regeneration column at the top as offgas together with the acidic gases which have been separated off, consisting mainly of carbon dioxide.

This leads to losses of hydrocarbons and in particular of olefins since these can only be separated from the acidic gas components with very great difficulty. Alkenes, in particular, dissolve very well in solvents which are used for the removal of acidic gases. If the hydrocarbon to be isolated is, for example, propylene, a significant part of the coabsorbed propylene is lost in the discharge process together with the carbon dioxide which has been removed in the regeneration of the solvent. Further recovery of propylene from this offgas is associated with high costs. This is economically disadvantageous for the overall process.

It is therefore an object of the invention to provide a process which allows improved separation of carbon dioxide and hydrocarbons in the acidic gas removal.

The invention achieves this object by means of a process which introduces a fuel gas into the bottom of a high-pressure flash vessel containing mass transfer elements such as random packing elements and ordered packing, where this gas is conveyed in counter-current to the solvent which is introduced at the top of the high-pressure flash vessel and also contains coabsorbed hydrocarbons and the major part of the hydrocarbons still present in the solvent is removed from the solvent and the carbon dioxide which is likewise present in the solvent is desorbed to only a small extent as a result of appropriate choice of temperature and pressure, and the fuel gas is the heating gas which is used for heating the dehydrogenation reactor and which is branched off. The desorption gas obtained from the high-pressure flash vessel is, in one embodiment of the invention, recirculated to the crude gas stream upstream of the compressor so that the recovered hydrocarbons are available in their entirety for the further product work-up.

As fuel gas, preference is given to using a hydrocarbon-containing gas such as natural gas. This is utilized for heating the dehydrogenation reactor and is used as branched-off substream for flow through the high-pressure flash vessel. The methane present as main component in the natural gas is discharged together with the hydrogen from the alkene isolation process and is used further as heating gas in the overall process.

The flow according to the invention of a fuel gas as stripping gas through the high-pressure flash vessel, which in a preferred embodiment of the invention is configured as a packed column, brings about a significant improvement in the desorption of the hydrocarbon obtained from the dehydrogenation reaction from the solvent for the gas scrub. In a preferred embodiment of the invention, the hydrocarbon obtained is recompressed and recirculated to the process. Carrying out the process of the invention enables the yield of olefin obtained from a dehydrogenation reaction to be increased significantly.

What is claimed is in particular a process for removing carbon dioxide from a hydrocarbon stream in the preparation of alkenes from alkanes, wherein
an industrially oxygen-free gas mixture containing alkanes and an inert gas is introduced into a reaction apparatus containing at least one catalyst bed to dehydrogenate the fed alkane and
the resulting gas mixture containing alkenes, alkanes, hydrogen, the inert gas and carbon dioxide (CO₂) is taken off from the reaction apparatus and
the acidic gas constituents such as carbon dioxide, hydrogen sulfide (H₂S), carbonyl sulfide (COS) and mercaptans still present in the process gas are completely or virtually completely absorbed by means of a chemical solvent in a gas scrub downstream of the dehydrogenation,

characterized in that
the adsorbate formed, which also contains coabsorbed hydrocarbons, is conveyed to the top of a high-pressure flash vessel containing mass transfer elements and a fuel gas is fed to the bottom of the high-pressure flash vessel and is conveyed in countercurrent to the enriched absorption solution flowing down over the mass transfer elements, with the major part of the hydrocarbons present in the enriched absorption solution and in particular the alkenes which are still dissolved being desorbed from the solution and leaving the high-pressure flash vessel at the top, and
the fuel gas is the heating gas which is used for heating the dehydrogenation reactor and which is branched off.

Since the stripping gas leaving the high-pressure flash vessel at the top is a hydrocarbon-containing gas having a composition similar to the process gas leaving the dehydrogenation apparatus, this can, in an advantageous embodiment of the invention, be used further as heating gas in the overall process in order to increase the efficiency.

The alkane used for the dehydrogenation is propane in a preferred embodiment. This is partly converted into propylene in accordance with the reaction equilibrium in the dehydrogenation apparatus. The feed gas used can also be n-butane, i-butane or n-pentane. n-Butene, i-butene or n-pentene are obtained as product hydrocarbons. In principle, any dehydrogenatable hydrocarbon can be used for the process.

The pressure in the high-pressure flash vessel is preferably greater than the pressure at the integration point at which the flash gas leaving the top of the “high-pressure flash vessel” is recirculated. A preferred integration point is the process gas path downstream of the dehydrogenation apparatus. The fuel gas for stripping is preferably natural gas which is also used as heating gas for the dehydrogenation.

The high-pressure flash vessel is operated at a pressure of from 1 to 20 bar. The high-pressure flash vessel is preferably operated at a pressure of from 2 to 12 bar and particularly preferably at a pressure of from 4 to 8 bar. The temperature of the solvent introduced at the top of the high-pressure flash vessel is set to a value in the range from 40° C. to 160° C., preferably to a temperature of from 80° C. to 120° C.

Suitable solvents for the gas scrub are solvents which are frequently used according to the prior art and have a good absorption capacity for carbon dioxide. Preference is given to chemically acting absorption media, in particular amine compounds such as monoethanolamine or methyldiethanolamine. These can be used as aqueous amine solution. They can also contain an activator. U.S. Pat. No. 6,290,754 B1 describes a process for deacidifying gases containing liquid hydrocarbons, in which the gas is treated with a liquid absorption medium containing methyldiethanolamine (MDEA) and an activator in an absorption zone so as to give a CO₂-rich gas fraction and a regenerated solvent stream and the regenerated solvent is recirculated to the absorption zone, with the activator being an aminoalkylamino-ethanol and the alkyl radical containing from 1 to 4 carbon atoms. This process and the solvent combination used therein can, by way of example, be used for the present process. It is possible to use any solvent which absorbs acidic gas components and is suitable for the process.

The solvent is taken off at the bottom of the high-pressure flash vessel and introduced into a solvent regeneration column. The solvent should first and foremost remove the acidic gas components such as hydrogen sulfide (H₂S), mercaptans, carbonyl sulfide (COS) and in particular carbon dioxide (CO₂) from the process gas selectively relative to the hydrocarbons. After desorption, these leave the solvent regeneration column at the top and can, after any necessary cooling, be reused or disposed of. It is possible to use all solvent regeneration columns known from the prior art which are suitable for a conventional solvent regeneration. These can, for example, be heated by means of a reboiler. After regeneration, the solvent is recirculated back to the absorption column; it can be conveyed via a heat exchanger to heat the loaded solvent before entry into the high-pressure flash vessel. If necessary, the regenerated solvent is cooled before reuse.

The purified product gas from the acidic gas scrub is fed to a “cold box” and a recovery column for further recovery of alkenes still present in the process gas. The separation is effected by means of low-temperature separation processes.

An apparatus for carrying out the process of the invention is also claimed. This comprises firstly an absorption column which makes possible a gas scrub by contacting with an acidic gas-absorbing solvent. The process of the invention is carried out using an apparatus which allows a fuel gas to be conveyed in countercurrent to the solution introduced at the top of the apparatus. This is preferably a high-pressure flash vessel which allows depressurization of the acidic gas-laden solvent and is provided with mass transfer elements, comparable to a packed column. The elements present in the high-pressure flash vessel can be mass transfer-enabling trays, packing elements, “Pall rings”, Raschig rings, structured packings or saddle bodies. The packed column contains an inlet for the acidic gas-laden solvent, an inlet for a fuel gas, an offtake for an acidic gas-laden solvent and an offtake for a gaseous hydrocarbon stream. The apparatus of the invention further comprises compressors, pumps, valves, pipes, control elements, filter devices and measurement devices, depending on requirements.

The invention enables acidic gases to be removed from a hydrocarbon stream, with the acidic gases being able to be freed virtually completely of hydrocarbons without a significantly increased outlay. The desorbed hydrocarbons can, in an advantageous embodiment, be recirculated to the process, as a result of which the efficiency of the process is increased.

The efficiency of the process is illustrated with the aid of an example. A gas stream to be purified which contains 47.92 kmol/h of water (H₂O), 1334.16 kmol/h of hydrogen (H₂), 32.26 kmol/h of nitrogen (N₂), 214.97 kmol/h of carbon dioxide (CO₂), 123.85 kmol/h of methane (CH₄), 89.14 kmol/h of ethane (C₂H₆), 2133.49 kmol/h of propane (C₃H₈), 1307.90 kmol/h of propene (C₃H₆) and 0.20 kmol/h of heavier hydro-carbons (C₄₊) was subjected to absorption with an amine solution as chemical absorption medium. In a subsequent desorption step, a gas containing 0.04 kmol/h of nitrogen, 0.59 kmol/h of carbon dioxide (CO₂) and 10.65 kmol/h of methane (CH₄, 88.9 mol percent) was passed through. A recovery of 98.4 mol percent of propene (C₃H₆) was achieved. The same gas was provided in the desorption with passage of a gas containing 0.10 kmol/h of nitrogen, 1.48 kmol/h of carbon dioxide (CO₂) and 26.64 kmol/h of methane (CH₄, 89.0 mol percent). A recovery of 99.8 mol percent of propene (C₃H₆) was achieved.

The configuration according to the invention of an apparatus for purifying an acidic gas-containing hydrocarbon stream is illustrated with the aid of a drawing, but the process of the invention is not restricted to this embodiment.

The gas (1) to be purified, preferably an alkene in admixture with hydrogen, alkanes, carbon dioxide, is introduced into an absorption column (2) in which the gas to be purified is brought into contact with an absorbing solvent (3) which has been fed in. This gives a gas stream (4) depleted in carbon dioxide. The solvent (5) enriched in carbon dioxide is taken off at the bottom of the column by means of a compressor (6), preheated by means of a heat exchanger (7) and depressurized into a high-pressure flash vessel (8) configured as a packed column. There, the loaded solvent is depressurized so that the absorbed gases are given off in the form of a hydrocarbon-rich gas stream (9). A fuel gas (10) which is a heating gas is conveyed in the opposite direction through the high-pressure flash vessel (8). The carbon dioxide-rich solvent (11) is obtained at the bottom of the high-pressure flash vessel (8) and is fed to a solvent regeneration column (12). The desorbed hydrocarbon-rich gas stream (9) is obtained at the top of the high-pressure flash vessel (8) in admixture with the fuel gas which is recirculated to the main process gas path via a compressor (13). At the top of the solvent regeneration column (12), the more or less pure carbon dioxide which has been driven off from the solvent is obtained as desorbed acidic gas (14). This is cooled by means of a suitable cooling device (14a) before reuse. The depressurized and desorbed solvent (12a) is obtained back at the bottom of the solvent regeneration column (12). The solvent regeneration column (12) is heated by means of a reboiler (12b) to effect desorption. The solvent is circulated (3) and cooled by means of a heat exchanger (7) for heating the starting solvent stream and a suitable cooling apparatus (3a) before reuse in the absorption column (2). The product stream (4) which has been depleted in carbon dioxide is fed to a cold box (not shown) for further product work-up.

### LIST OF REFERENCE NUMERALS

1 Gas to be purified
2 Absorption column
3 Carbon dioxide-absorbing solvent
3a Solvent cooler
4 Gas stream depleted in carbon dioxide
5 Solvent enriched in carbon dioxide
6 Compressor
7 Heat exchanger
8 “High-pressure flash vessel” (packed column)
9 Hydrocarbon-rich gas stream
10 Fuel gas
11 Carbon dioxide-rich solvent
12 Solvent regeneration column
12a Desorbed solvent
12b Reboiler
13 Compressor
14 Desorbed acidic gas
14a Condenser

1.-16. (canceled)

17. A process for removing carbon dioxide from a hydrocarbon stream in the preparation of alkenes from alkanes, comprising:
introducing an industrially oxygen-free gas mixture containing a feed alkane and an inert gas into a reaction apparatus containing at least one catalyst bed to dehydrogenate the feed alkane, said reaction apparatus heated by a heating gas;
removing from the reaction apparatus a product gas mixture containing alkenes, alkanes, hydrogen, the inert gas and carbon dioxide (CO₂);
absorbing acidic gas constituents comprising one or more of carbon dioxide, hydrogen sulfide (H₂S), carbonyl sulfide (COS) and mercaptans present in the product gas by contacting the product gas with a solvent which absorbs acidic gases in a gas scrub downstream of the dehydrogenation, forming an adsorbate solution; and
conveying the adsorbate solution, which also contains coabsorbed hydrocarbons, to the top of a high-pressure flash vessel containing mass transfer elements, and feeding a fuel gas to the bottom of the high-pressure flash vessel countercurrent to the adsorbate solution flowing downwards over the mass transfer elements, wherein the major part of hydrocarbons present in the adsorbate solution including alkenes desorbed from the adsorbate solution leave the high-pressure flash vessel at the top,
wherein the fuel gas is the heating gas which is used for heating the dehydrogenation reactor.

18. The process of claim 17, wherein gas leaving the top of the high-pressure flash vessel is recirculated to the product gas path downstream of the dehydrogenation apparatus.

19. The process of claim 17, wherein the high-pressure flash vessel is operated at a pressure of from 1 to 20 bar.

20. The process of claim 17, wherein the high-pressure flash vessel is operated at a temperature of from 40° C. to 160° C.

21. The process of claim 17, wherein the alkane is propane and the alkene prepared therefrom is propylene.

22. The process of claim 17, wherein the inert gas in the dehydrogenation reaction is steam.

23. The process of claim 22, wherein the heating gas used for heating the dehydrogenation reactor is natural gas.

24. The process of claim 17, wherein the solvent for the gas scrub comprises a chemically acting absorption medium.

25. The process of claim 17, wherein the solvent for the gas scrub comprises an aqueous amine solution.

26. The process of claim 17, wherein an aqueous monoethanolamine solution is used as a solvent for the gas scrub.

27. The process of claim 17, wherein the solvent for the gas scrub comprises an aqueous methyldiethanolamine solution.

28. The process of claim 26, wherein the solvent for the gas scrub contains an activator selected from the group consisting of primary and secondary amines.

29. The process of claim 27, wherein the solvent for the gas scrub contains an activator selected from the group consisting of primary and secondary amines.

30. The process of claim 17, wherein the product gas which has been freed of acidic gases is fed to a “cold box” and to a recovery column for further recovery of alkenes still present in the product gas.

31. An apparatus for removing carbon dioxide from a hydrocarbon stream in the preparation of alkenes from alkanes, comprising a gas scrubbing absorption column which contacts an acidic gas-containing process stream with an acidic gas-absorbing solvent, wherein downstream of the absorption column there is a high-pressure flash vessel which depressurizes acidic gas-laden solvent and this high-pressure flash vessel has an inlet for the acidic gas-laden solvent, an inlet for a fuel gas, an outlet for an acidic gas-laden solvent and an outlet for a gaseous hydrocarbon stream.
